(19) United States
(12) Patent Application Publication
(10) Pub. No.: US 2007/0239173 A1
(43) Pub. Date: Oct. 11, 2007

(54) Hydro-dermabrasion apparatus and method of use

(76) Inventor: Ben M. Khalaj, San Clemente, CA (US)

Correspondence Address: Ben M. Khalaj, 106 Arista, San Clemente, CA, 92673, US

(21) Appl. No.: 11/393,068

(22) Filed: Mar. 30, 2006

(51) Int. Cl. A61B 17/50

(52) U.S. Cl. 606/131

(57) ABSTRACT

It is an object of the present invention to provide a crystal free dermabrasion applicator with water based abraded tip. The applicator is using a vacuum source to remove debris and to resurface skin via its abraded tip. The waste and debris of the skin and abrasion particles will be collected by a filter in a collection canister. The collection canister also has a reservoir for collecting the diluted water used in dermabrasion process.

### FIELD OF THE INVENTION

The present invention relates to skin care using dermabrasion and more specifically to crystal free and water based dermabrasion applicator.

### BACKGROUND OF THE INVENTION

The desire to improve the skin through non-surgical means has lead to the increase in popularity of micodermabrasion over the years. Microdermabrasion and dermabrasion system improves the skin by removing the dead skin cells from the top layer of the skin, thus reducing the appearance of fine lines and wrinkles, age spot, and acne scaring, evening out skin tone, and promote the growth of healthy new skin cells. This is all achieved through gentle abrasion of the skin using a device combined with vacuum suction and crystal flow. For those who want microdermabrasion without the crystal, an alternative does exist; crystal free dermabrasion.

Some prior patents refer to crystal free dermabrasion. For example, U.S. Pat. No. 6,629,983 B1, issued to Roger G. Ignon et al, entitled “Apparatus and method for skin/surface abrasion” disclosing “A dermabrasion handpiece adapted for use to abrade the skin of a patient includes a housing extending along an axis between a proximal end and a distal end . . . . The housing is adapted for connection to a vacuum source to pull a portion of the skin through the hole and to move the skin portion into contact with abrasion element.”

In spite of the advantages of the devices in prior art, there are some limitations which cannot be delivered by using a dry process in conjunction with crystal free dermabrasion processes.

This invention overcomes the shortcomings of conventional devices by providing a water based crystal free dermabrasion using an applicator with a replaceable abraded tip that can abrade skin and resurface the skin and provide a healthy skin thereof.

### SUMMARY OF THE INVENTION

It is an object of the present invention to provide a crystal free dermabrasion applicator with water based abraded tip. The applicator is using a vacuum source to remove debris and to resurface skin via its abraded tip. The waste and debris of the skin and abrasion particles will be collected by a filter in a collection canister. The collection canister also has a reservoir for collecting the diluted water used in dermabrasion process. This hydro-dermabrasion process eliminates dry processes of abrasion media used on skin by prior art devices. Therefore, it reduces friction and unnecessary heat applied by abrasion media on skin and furthermore provides cooler process and better finishing of the skin.

It is another object of this invention to provide a method of using the hydro-dermabrasion applicator with its disposable tip. In this method a conventional wet sponge is used to moisten the abraded tip as needed for dermabrasion process.

It is yet another object of this invention to provide another method of using the hydro-dermabrasion applicator with its disposable tip having a port for direct delivery of water supply. In this method water is delivered on skin as the abraded tip is applied on skin.

It is yet another object of this invention to provide a disposable abraded tip with an option of having different grade of grits.

Further objects and advantages of this invention will become apparent from consideration of the drawings and descriptions that follow.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the invention showing the invention is adapted in conjunction with a microdermabrasion system.

FIG. 2 is a perspective view of the hydro-dermabarsion apparatus showing the present invention.

FIG. 3 is a partial cross section of the applicator shown in FIG. 1 and FIG. 2.

FIG. 4 is an alternative partial cross section of the applicator shown in FIG. 3.

FIG. 5 is a perspective view of the disposable tip of the applicator.

FIG. 6 is an exploded perspective view of disposable tip shown in FIG. 5.

FIG. 7 is alternative perspective view of FIG. 5.

FIG. 8 is a diagram shown the method of using the applicator with its disposable tip.

### DETAILED DESCRIPTION OF THE INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

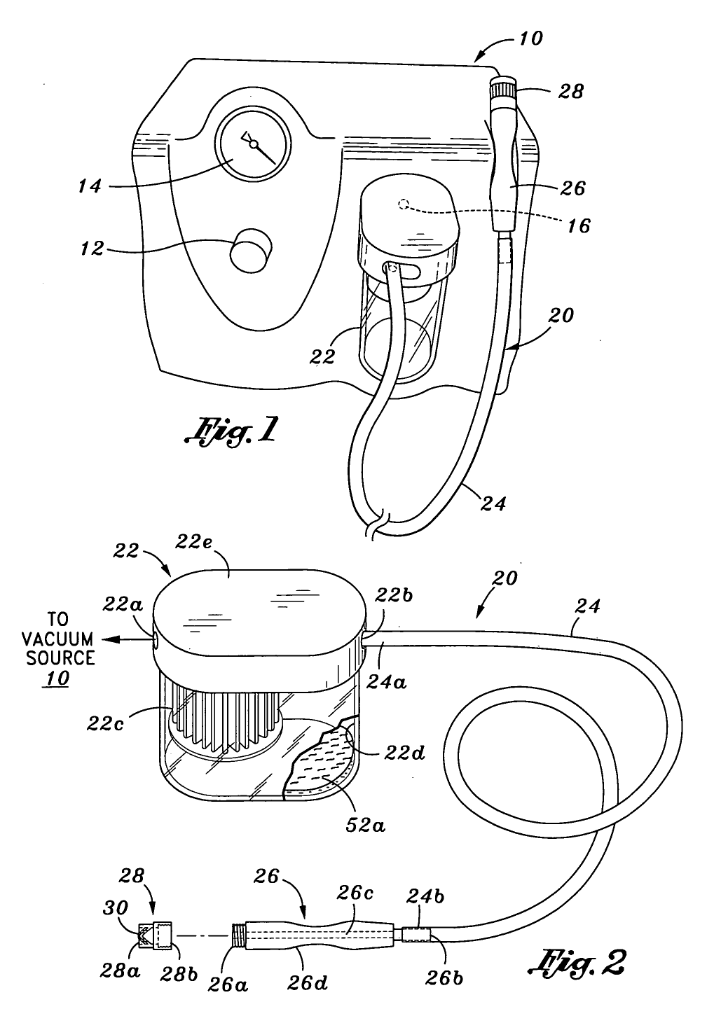

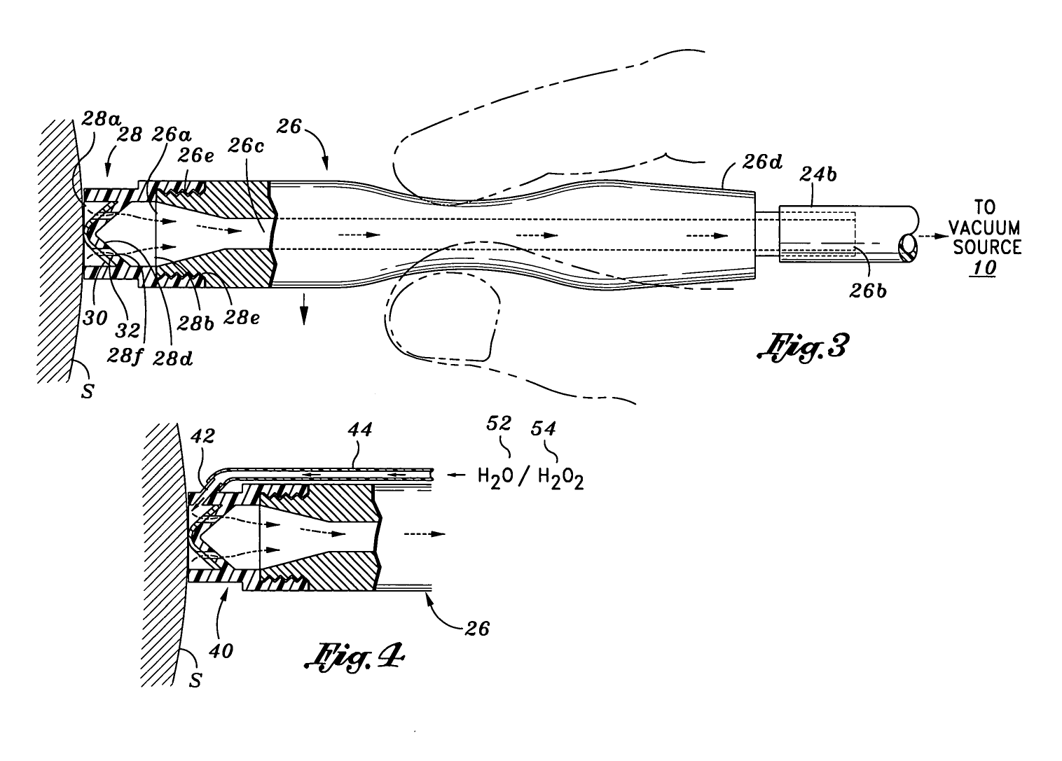

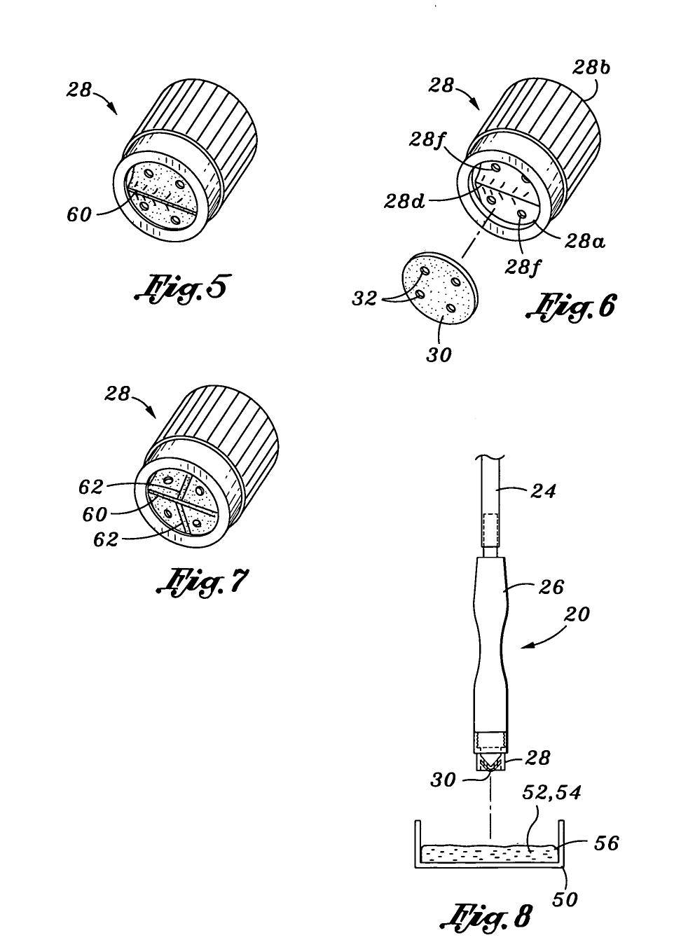

FIG. 1 is a perspective view of a microdermabrasion 10 system in conjunction with present invention showing a hydro-dermabrasion applicator assembly 20. The applicator assembly includes a handpiece 26 that has a central axial lumen 26c and a abraded tip 28 at its proximal end. The handpiece 26 is connected to a collection canister 22 via a hollow tube 24 at its distal end. The canister 22 is connected to a vacuum source of microdermabrasion system via a vacuum port 16. The applied vacuum source can be adjusted by a knob 12 and can be measured by a gauge 14. The collection canister 22 can receive the suction from vacuum pump to its receiving port 22a as seen in FIG. 2. The canister 22 includes a replaceable filter 22c and a reservoir 22d to collect debris and diluted water 52a. When the suction is applied at the handpiece tip, the abraded tip pulls the skin against the folded edge of the tip that has disk grit. The abraded tip 28 is a disposable item which is connected via internal threads 28e to the proximal end of the handpiece threads 26e as shown in FIG. 3. The abraded tip includes a grit disk 30 that glued into the tip's internal body at its proximal end. The grit disk 30 is flexible part with and preferably made of silicon carbide material on exterior side and has adhesive on opposite side. Disk grits also can come to in different grad of grits for purpose of abrasion process. When the flexible grit disk is placed into the tip, the disk is folded in a way to create contacting edges 60, 62 and its conforms to the internal folds 28d of the tip's body as illustrated in FIG. 5-7. Creation of these edges 60, 62 provides a better contact to the skin when moving the applicator in up and down direction. The grit disk 30 and the internal fold 28d of the tip 28 have passage holes 32, 28f to remove the debris and waste material from skin by applied suction from the vacuum source. Referring to FIG. 8, a first method of using the applicator in conjunction with wet abrasion is disclosed. In this view the applicator tip 28 is dipped into a moisten sponge 56 and the wet grit 32 is applied on skin by moving the applicator in desired direction to remove the dead skin cells and bring out the healthy skin. The applicator tip 28 can repetitively be dipped into the moisten sponge 56 when needed. The sponge 56 is placed in a reservoir 50 with a supply of water (H2O) 52 or oxygenated water (H2O2) 54.

The second method of using the applicator in conjunction with wet abrasion is shown in FIG. 4. In this view, the abraded tip 40 includes a port 42 for receiving water over the abraded tip. A tube 44 is connected to port 42 of the tip to provide water supply for dermabrasion as needed.

While this invention is susceptible of embodiments in many different forms, this specification and the accompanying drawings disclose only some specific forms as examples of the invention. The invention is not intended to be limited to the embodiments so described; however, the scope of the invention is pointed out in the appended claims.

I claim:

1. A hydro-dremabrasion apparatus to be used in conjunction with vacuum source comprising:
a handpiece, said handpiece has a central axial lumen for passage of vacuum suction, a distal end and a proximal end,
a replaceable abraded tip, said abraded tip connected to proximal end of said handpiece, said abraded tip includes a grit disk at its proximal end and positioned into said tip's body in a folded way in order to create pointed edges,
a collection canister, said collection canister at one end connected to vacuum source and on the other end connected to distal end of the handpiece via a vacuum tube, said collection canister collect debris and waste from dermabrasion process applied on skin, and;
means to provide water supply to said abraded tip for wet abrasion process.

2. A hydro-dremabrasion apparatus according to claim 1, wherein said abraded tip is disposable and can be replaced by different grade grit if necessary.

3. A hydro-dremabrasion apparatus according to claim 1, wherein said grit disk is flexible and can conform to said tip's internal body.

4. A hydro-dremabrasion apparatus according to claim 1, wherein said grit disk made of silicon carbides material.

5. A hydro-dremabrasion apparatus according to claim 1, wherein said collection canister includes a replaceable filter and a reservoir.
